(19) United States
(12) Patent Application Publication
(10) Pub. No.: US 2019/0365334 A1
(43) Pub. Date: Dec. 5, 2019

(54) HEALTH NOTIFICATION SYSTEM AND METHOD

(71) Applicant: Joshua Railton M. Blanchard, Baldwin (CA)

(72) Inventor: Joshua Railton M. Blanchard, Baldwin (CA)

(21) Appl. No.: 15/991,949

(22) Filed: May 29, 2018

(51) Int. Cl. A61B 5/00; A61B 5/024; G08B 21/04

(52) U.S. Cl. CPC A61B 5/7465; G08B 21/0269; G08B 21/0453; A61B 5/02438

(57) ABSTRACT

A health notification system includes a wearable device for monitoring a vital sign of a user, a mobile device communicably coupled to the wearable device, and a software interface configured to operate a mobile application being located on the mobile device. The wearable device further includes a wireless communication module configured to communicate with the mobile device. Upon notification that the wearable device has detected a specified condition, the mobile application executes a message trigger function established to deliver at least one preconfigured message to a recipient or recipients.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of monitoring systems of existing art and more specifically relates to software for communicating with a health monitor.

### RELATED ART

A person's safety and security is often a concern to one's friends and family. Existing health and safety monitoring devices and systems are inadequate regarding cost, efficacy, and portability. Further, most existing health monitoring devices and systems are simply configured to function while the user is in good health or to notify when health concerns are present.

Unfortunately, users of such systems end up dying suddenly, thereby rendering the health monitoring system useless before the user has a chance to communicate one last time with friends, family members, or loved ones. Some users choose to keep a last will and testament or final notices to family members, but these messages and notices are often delayed for delivery or do not end up being delivered in the manner intended. A suitable solution is desired.

U.S. Pat. No. 9,572,503 to Eric DeForest relates to a personal safety and security mobile application responsive to changes in heart rate. The described personal safety and security mobile application responsive to changes in heart rate includes a software app for a mobile device is disclosed for alerting a custodian of a person to be protected of an emergency situation involving the person to be protected. The app includes software instructions for carrying out a method including: establishing a range of normal heart rates for the person using a heart rate monitor; detecting a heart rate for the person that is outside of the established range; activating at least one of a camera, a microphone, an accelerometer, and a location indicator on the mobile device carried by the person; establishing a wireless data connection between the mobile device and a communication network; and transmitting data to the custodian from the activated camera, microphone, accelerometer, or location indicator via the communication network. The app can notify a custodian of a medical or safety emergency as it is happening, giving that custodian the ability to immediately notify emergency personnel.

### SUMMARY OF THE INVENTION

In view of the foregoing disadvantages inherent in the known monitoring systems art, the present disclosure provides a novel health notification system. The general purpose of the present disclosure, which will be described subsequently in greater detail, is to provide a wearable monitor and software application suited to track a user's heart rate and deliver a plurality of notifications to multiple recipients upon notification that specified thresholds have been triggered.

A health notification system is disclosed herein. The health notification system may include a wearable device for monitoring a vital sign of a user, a mobile device communicably coupled to the wearable device, and a software interface configured to operate a mobile application being located on the mobile device. The wearable device may further include a wireless communication module configured to communicate with the mobile device. Upon notification that the wearable device has detected a specified condition, the mobile application may execute a message trigger function established to deliver at least one preconfigured message to a recipient or recipients.

According to another embodiment, a method of using a health notification system is also disclosed herein. The method of using a health notification system may include providing a health notification system including a wearable device for monitoring a vital sign of a user, the wearable device further including a wireless communications module, a mobile device communicably coupled to the wearable device, a software interface configured to operate a mobile application being located on the mobile device, and a message trigger function contained within the mobile application, programming a death message to be delivered by the message trigger function, and configuring a message delay setting.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a health notification system, constructed and operative according to the teachings of the present disclosure.

FIG. 1 is a perspective view of the health notification system during an ‘in-use’ condition, according to an embodiment of the disclosure.

FIG. 2 is a front view of the health notification system of FIG. 1, according to an embodiment of the present disclosure.

FIG. 3 is a top perspective view of the wearable device of the health notification system of FIG. 1, according to an embodiment of the present disclosure.

FIG. 4 is a perspective view of a mobile device configured to operate the mobile application of the health notification system of FIG. 1, according to an embodiment of the present disclosure.

FIG. 5 is a flow diagram illustrating a method of using a health notification system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a monitoring system and more particularly to a health notification system as used to provide a wearable monitor and software application suited to track a user's heart rate and deliver a plurality of notifications to multiple recipients upon notification that specified thresholds have been triggered.

Generally, the aforementioned system may include two main components: the first component may include a wearable wristband that can monitor heart rate and transmit data to an app on a smart phone. The purpose of the heart monitor is to send an alert to the app when the heart rate drops to zero beats/minute; the second component may include an app that will monitor the heart rate and send a delayed text, video or voice messages upon the event that no heart rate is detected and in the event of death. The app will may also have the ability to connect to other wearable devices such as existing third-party products to obtain heart rate information.

The included software application may include a feature to send a message of a recording or written message to whomever the user has selected in their contact lists. These messages may be configured to be sent upon the user's heartbeat reaching zero beats per minute. The preconfigured message may be setup to be delivered on a time-delayed format, so that a user's loved ones are not finding out about the passing of the user through the app, but rather the message may be delivered after some time has gone by so that the loved ones may have a final message to remember the user by.

The wearable device of this system may be configured as a wristband consisting of a a wireless communications module for sending and/or receiving short-range wireless signals, a hear rate monitor that allows for tracking of user vital signs including a pulse, and a global positioning apparatus or system to track the location of the user. The wearable device may be communicably coupled to a mobile device, wherein the mobile device includes a software interface configured to operate a mobile application. The mobile application may include a message trigger function that allows for sending at least one message to a predetermined recipient after a condition has been satisfied. This condition may be when a user's pulse reaches zero beats per minute, at which time a death message may be sent via the message trigger function. This death message may be in the form of an audio message, written message, or other type of communication. The death message may be delivered after a time delay has elapsed. The death message may not necessarily be suited for informing friends or family of the passing of a user, but rather to send a message of comfort and remembrance.

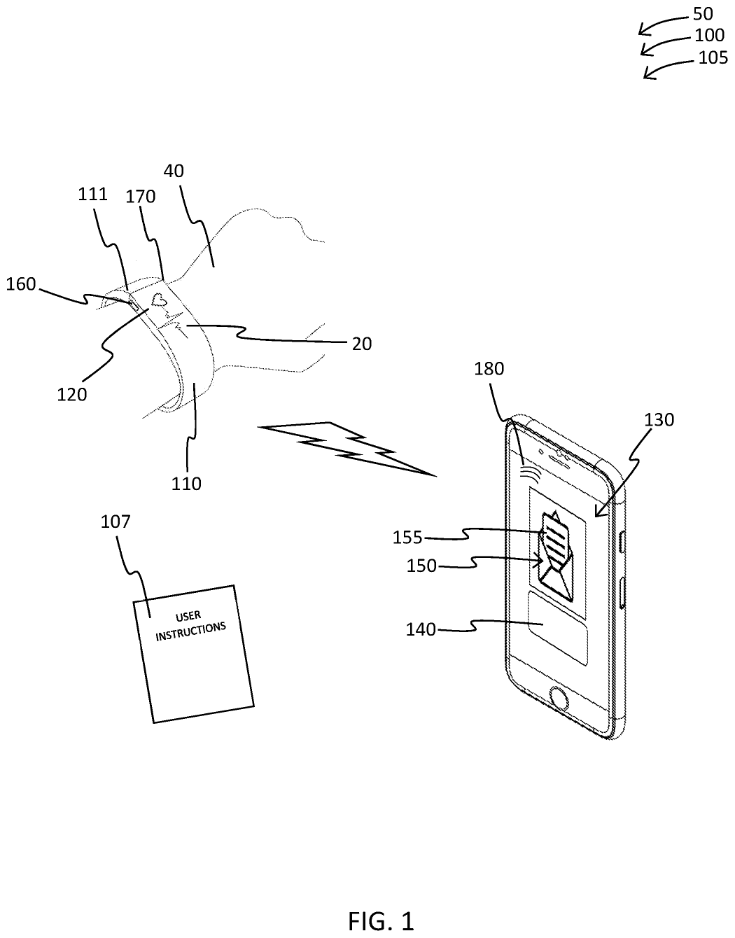

Referring now more specifically to the drawings by numerals of reference, there is shown in FIGS. 1-4, various views of a health notification system 100.

FIG. 1 shows a health notification system 100 during an ‘in-use’ condition 50, according to an embodiment of the present disclosure. Here, the health notification system 100 may be beneficial for use by a user 40 to provide a wearable monitor and software application suited to track a user's heart rate and deliver a plurality of notifications to multiple recipients upon notification that specified thresholds have been triggered. As illustrated, the health notification system 100 may include a wearable device 110 for monitoring a vital sign 20 of a user 40. The wearable device 110 may be configured as a wristband 111 for wearing around a wrist of the user 40. The wearable device 110 may further include a wireless communications module 120 that allows for sending and/or receiving a short-range wireless signal. In addition, the wearable device 110 may feature a global positioning system 160 to track a location of the user 40 and a heart rate monitor 170. The wearable device 110 may be communicably coupled to a mobile device 130 that includes a software interface 140 configured to operate a mobile application 150 located on said mobile device. The mobile application 150 may include a message trigger function 155 and a wireless communication protocol 180 for executing said message trigger function 155.

According to one embodiment, the health notification system 100 may be arranged as a kit 105. In particular, the health notification system 100 may further include a set of instructions 107. The instructions 107 may detail functional relationships in relation to the structure of the health notification system 100 such that the health notification system 100 can be used, maintained, or the like, in a preferred manner.

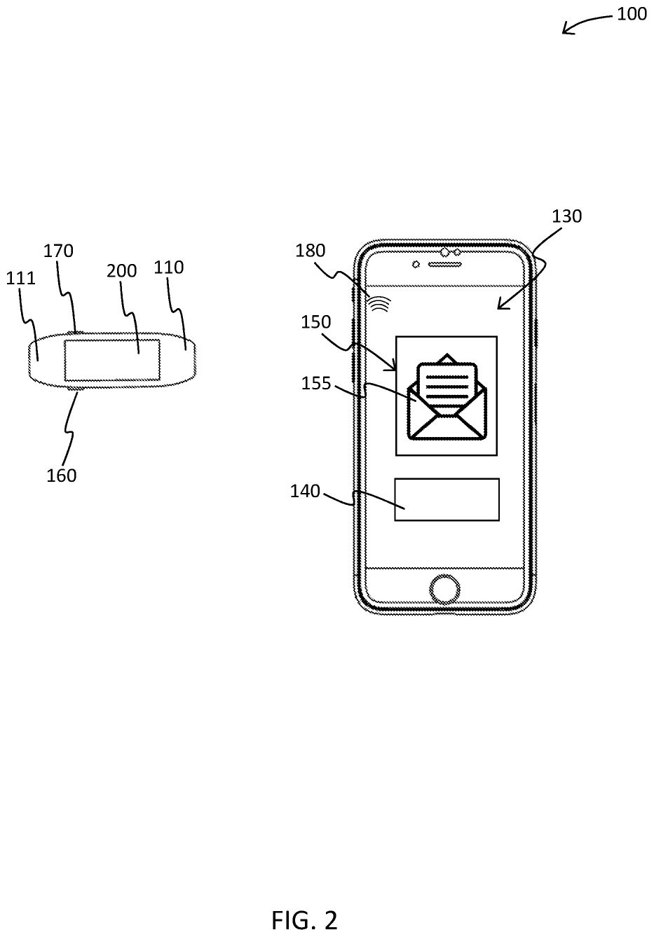

Referring now to FIG. 2 showing the health notification system 100 of FIG. 1, according to an embodiment of the present disclosure. As above, the health notification system 100 may include a wearable device 110 that may be configured as a wristband 111. The wearable device 110 may feature a global positioning system 160 (or other suitable means) to track a location and a heart rate monitor 170. Here again, the health notification system 100 may include a wearable device 110 that may be communicably coupled to a mobile device 130 that includes a software interface 140 configured to operate a mobile application 150 located on said mobile device. The mobile application 150 may include a message trigger function 155 and a wireless communication protocol 180 for executing said message trigger function 155. The wireless communication protocol 180 may further be configured to interact with a secondary wearable device 200 from a manufacturer outside of the current health notification system 100.

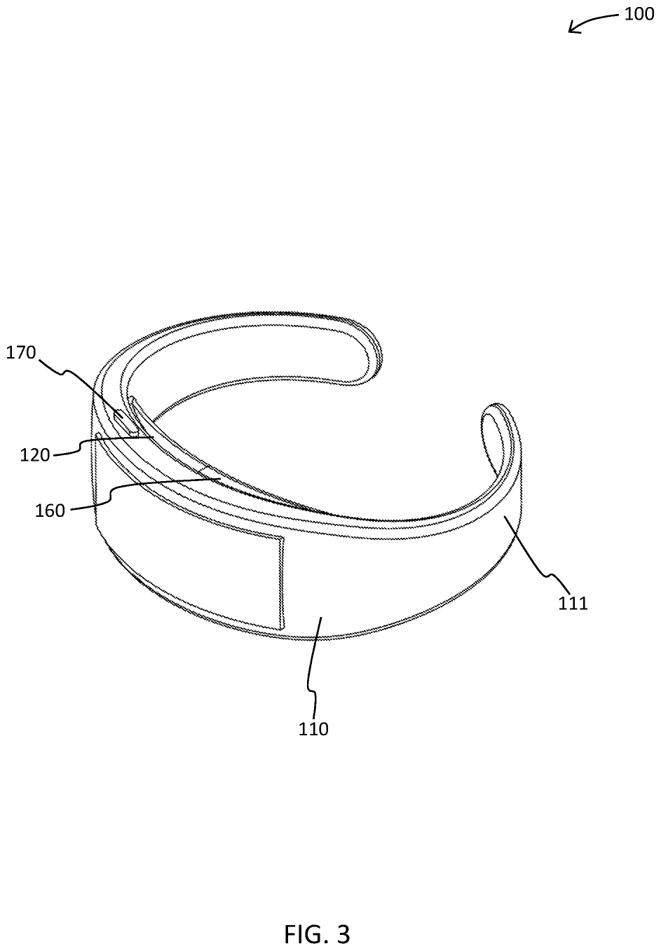

FIG. 3 is a top perspective view of the wearable device 110 of the health notification system 100 of FIG. 1, according to an embodiment of the present disclosure. As above, the health notification system 100 may include a wearable device 110 that may be configured as a wristband 111. The wearable device 110 may feature a global positioning system 160 to track a location and a heart rate monitor 170. The wearable device 110 may further include a wireless communications module 120 that allows for sending and/or receiving a short-range wireless signal.

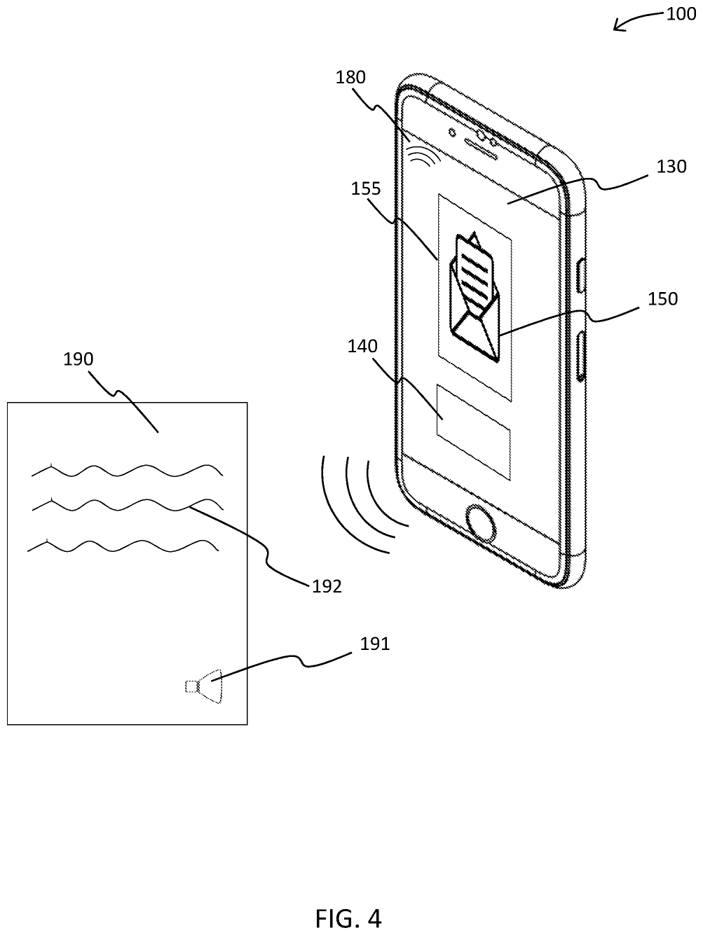

Referring now to FIG. 4, a perspective view of the health notification system 100 of FIG. 1, according to an embodiment of the present disclosure. Here again, the health notification system 100 may include a wearable device 110 (see FIG. 1) that may be communicably coupled to a mobile device 130 that includes a software interface 140 configured to operate a mobile application 150 located on said mobile device. The mobile application 150 may include a message trigger function 155 and a wireless communication protocol 180 for executing said message trigger function 155. The message trigger function 155 may allow a death message 190 to be sent to intended recipients after a predetermined time has passed following the death of a user. The death message 190 may consist of an audio recording 191 and/or a written notification 192.

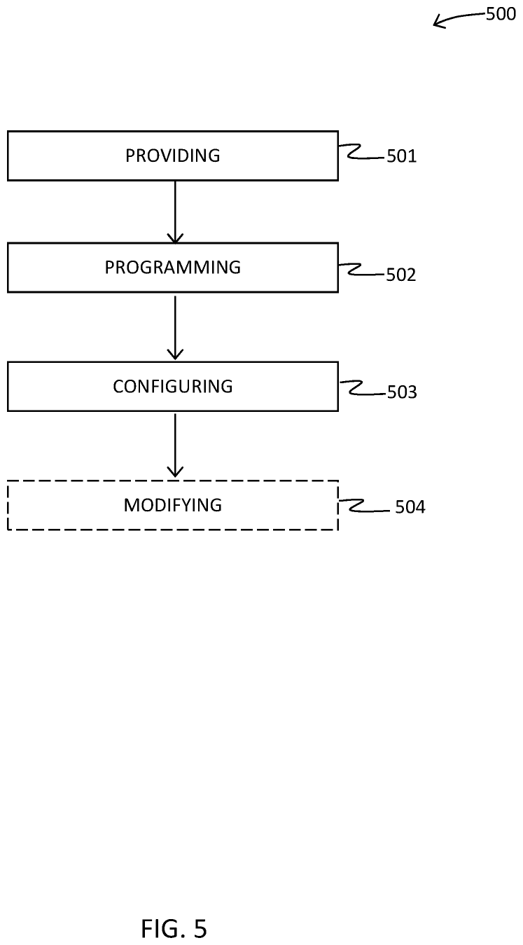

FIG. 5 is a flow diagram illustrating a method of using a health notification system 500, according to an embodiment of the present disclosure. In particular, the method of using a health notification system 500 may include one or more components or features of the health notification system 100 as described above. As illustrated, the method of using a health notification system 500 may include the steps of: step one 501, providing a health notification system including a wearable device for monitoring a vital sign of a user, the wearable device further including a wireless communications module, a mobile device communicably coupled to the wearable device, a software interface configured to operate a mobile application being located on the mobile device, and a message trigger function contained within the mobile application; step two 502, programming a death message to be delivered by the message trigger function; and step three 503, configuring a message delay setting and optionally step four 504 modifying the death message and/or message delay setting.

It should be noted that step 504 is an optional step and may not be implemented in all cases. Optional steps of the method of using a health notification system 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for health notification system 100, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A health notification system comprising:
a wearable device for monitoring a vital sign of a user, the wearable device further including a wireless communications module;
a mobile device communicably coupled to the wearable device;
a software interface configured to operate a mobile application being located on the mobile device; and
a message trigger function contained within the mobile application;
wherein the mobile application is configured to receive the at least one vital sign and respond to a set of user commands via the message trigger function when a condition has been triggered.

2. The health notification system of claim 1, wherein the wearable device is configured as a wristband for wearing around a wrist of the user.

3. The health notification system of claim 1, wherein the wearable device further includes a global positioning system to track a location of the user.

4. The health notification system of claim 1, wherein the wearable device further includes a heart rate monitor.

5. The health notification system of claim 4, wherein the vital sign includes a pulse of the user as determined by the heart rate monitor.

6. The health notification system of claim 1, wherein the wireless communications module is configured to send a short-range wireless signal.

7. The health notification system of claim 1, wherein the mobile application further includes a wireless communication protocol for executing the message trigger function.

8. The health notification system of claim 1, wherein the condition is satisfied when the pulse of the user reaches zero beats per minute.

9. The health notification system of claim 1, wherein the wireless communication protocol is further configured to interact with a secondary wearable device.

10. The health notification system of claim 9, wherein the secondary wearable device includes a wearable monitor from a secondary manufacturer.

11. The health notification system of claim 1, wherein the set of user commands includes a death message configured for distribution to at least one recipient designated by the user.

12. The health notification system of claim 11, wherein the death message includes an audio recording.

13. The health notification system of claim 11, wherein the death message includes a written notification.

14. The health notification system of claim 1, wherein the set of user commands includes a message delay setting.

15. The health notification system of claim 11, wherein the message delay setting is configured to withhold the death message until a specified time has elapsed following the condition being satisfied.

16. The health notification system of claim 15, wherein the specified time is selected by the user.

17. A health notification system comprising:
a wearable device for monitoring a vital sign of a user, the wearable device further including a wireless communications module;
a mobile device communicably coupled to the wearable device;
a software interface configured to operate a mobile application being located on the mobile device; and
a message trigger function contained within the mobile application;
wherein the mobile application is configured to receive the at least one vital sign and respond to a set of user commands via the message trigger function when a condition has been triggered;
wherein the wearable device is configured as a wristband for wearing around a wrist of the user;
wherein the wearable device further includes a global positioning system to track a location of the user;
wherein the wearable device further includes a heart rate monitor;
wherein the vital sign includes a pulse of the user as determined by the heart rate monitor;
wherein the wireless communications module is configured to send a short-range wireless signal;
wherein the mobile application further includes a wireless communication protocol for executing the message trigger function;
wherein the condition is satisfied when the pulse of the user reaches zero beats per minute;
wherein the wireless communication protocol is further configured to interact with a secondary wearable device;
wherein the secondary wearable device includes a wearable monitor from a secondary manufacturer;
wherein the set of user commands includes a death message configured for distribution to at least one recipient designated by the user;
wherein the death message includes an audio recording;
wherein the death message includes a written notification;
wherein the set of user commands includes a message delay setting;
wherein the message delay setting is configured to withhold the death message until a specified time has elapsed following the condition being satisfied; and
wherein the specified time is selected by the user.

18. The health notification system of claim 17, further comprising set of instructions; and
wherein the health notification system is arranged as a kit.

19. A method of using a health notification system, the method comprising the steps of:
providing a health notification system including a wearable device for monitoring a vital sign of a user, the wearable device further including a wireless communications module, a mobile device communicably coupled to the wearable device, a software interface configured to operate a mobile application being located on the mobile device, and a message trigger function contained within the mobile application;
programming a death message to be delivered by the message trigger function; and
configuring a message delay setting.

20. The method of claim 19, further comprising the steps of:
modifying the death message and/or message delay setting.
